(19) United States
(12) Patent Application Publication
(10) Pub. No.: US 2024/0296949 A1
(43) Pub. Date: Sep. 5, 2024

(54) DIGITAL CELL PATHOLOGY DISPLAY AND CLASSIFICATION METHODS AND SYSTEM

(71) Applicant: VisionGate, Inc., Woodinville, WA (US)

(72) Inventors: Michael G. Meyer, Phoenix, AZ (US); David Wilbur, Woodinville, WA (US); Chris Presley, Woodinville, WA (US); Celeste Hamilton, Woodinville, WA (US); Rahul Katdare, Woodinville, WA (US); Greg Zick, Woodinville, WA (US); Frances Lakers, Woodinville, WA (US)

(21) Appl. No.: 18/503,002

(22) Filed: Nov. 6, 2023

Related U.S. Application Data
(60) Provisional application No. 63/423,433, filed on Nov. 7, 2022.

(51) Int. Cl. G16H 50/20; G01N 15/14; G01N 15/1434; G01N 33/483; G16H 10/60; G16H 30/40

(52) U.S. Cl. CPC G16H 50/20; G01N 15/1434; G01N 33/4833; G16H 10/60; G16H 30/40; G01N 2015/1402; G01N 2015/1445

(57) ABSTRACT

A cell classification system includes an optical tomography system and a processor operable to generate a plurality of 2D images and a plurality of pseudo-projection images of a cell. The processor executes instructions that cause the processor to: generate a 3D image of the cell using the pseudo-projection images; apply digital enhancement to a 2D or 3D image to improve determination of boundaries of structures with the cell that provide indicia of cell features; analyze at least one of the cell features or 2D or 3D images using AI-based cell characterization to characterize the cell as normal or as having abnormal features by analyzing the boundaries of structures; create a Normal Cell Gallery comprising images characterized by AI-based characterization as normal; create a Diagnostic Cell Gallery with images characterized by AI-based characterization as having abnormal features; display at least the Diagnostic Cell Gallery; and record a user classification.

### TECHNICAL FIELD

The present disclosure relates to a system and method for digital cell pathology, for example, to display normal cells and cells that have abnormal features that may be indicative of cancer.

### BACKGROUND

Traditional cell pathology methods allow examination of cells using a microscope and thus, provide only two-dimensional (2D) views of the cell from a single orientation, with only stains and other physical techniques available for image enhancement. In addition, traditional cell pathology methods make it difficult to compare multiple cells and retain cells' images for records. Traditional cell pathology methods are also limited in that the pathologist must have the sample in their physical presence to analyze the sample.

### BRIEF SUMMARY

The present disclosure relates to a system and method for digital cell pathology to display normal cells and cells with abnormal features that may indicate cancer. Briefly stated, one or more embodiments, a digital cell pathology method is disclosed that includes: (a) generating, by an optical tomography system, a plurality of 2D images of a cell from a patient sample comprising a plurality of cells; (b) generating, by the optical tomography system, a plurality of pseudo-projection images of the cell; (c) generating a 3D image of the cell using the pseudo-projection images; (d) applying digital enhancement to at least one of the 2D or 3D images to improve determination of boundaries of structures with the cell, wherein the boundaries of structures with the cell provide indicia of abnormal features; (e) analyzing at least one of the 2D or 3D images by AI-based cell characterization to characterize the cell as normal or as having abnormal features by (i) analyzing features pre-determined by a human, wherein the analysis includes analyzing the boundaries of structures with the cell, (ii) analyzing features determined by AI or other image components, or (iii) both; (f) increasing a total cell count number by one; (g) determining if total cell count number has reached a pre-selected number and, if not, returning to step a); (h) if the total cell count number has reached the pre-selected number, providing images of the cell to a user, wherein providing images comprises: creating a Normal Cell Gallery comprising images of cells characterized by AI-based characterization as normal, creating a Diagnostic Cell Gallery with images of cells characterized by AI-based characterization as having abnormal features, and displaying at least the Diagnostic Cell Gallery to the user; and (i) recording a user classification for at least one cell characterized by AI-based characterization as having abnormal features.

In some embodiments of the method for digital cell pathology, the digital enhancement includes one or more of image sharpening, segmentation, contrast, scale bar overlay, brightness, gamma, color transformation, and opacity transformation. In another aspect of some embodiments, the analysis of the at least one of the 2D or 3D images by AI-based cell characterization includes aiding cell interpretation using patient age, patient gender, patient prior history with cancer, and patient prior history with non-cancer diseases. In still another aspect of some embodiments, the method for digital cell pathology further includes using the recorded user classification and data regarding the patient from whom the patient sample was obtained to generate an abnormality index for the patient sample. In yet another aspect of some embodiments, the method for digital cell pathology further includes comparing the abnormality index to a cancer threshold value and identifying the patient sample as positive or negative for cancer based upon the comparison.

In one or more embodiments of the method for digital cell pathology, the AI-based cell characterization has a pre-selected sensitivity of at least 85%. In another aspect of some embodiments, the AI-based cell characterization has a pre-selected specificity of at least 60%. In still another aspect of some embodiments, the 3D image has isotrophic resolution. In yet another aspect of some embodiments, the 3D image has a resolution to a distance smaller than a wavelength of light used to generate the pseudo-projection images. Furthermore, in some embodiments, the 3D image has a resolution of 200 nm.

In some embodiments of the method for digital cell pathology, the at least one cell feature in the 3D image of a cell characterized by AI-based characterization as having abnormal features is identified by and highlighted in the 3D image by a processor. In another aspect of some embodiments, the at least one cell feature is highlighted by use of different colors for different cell features, decreasing the opacity of surrounding cell features, or both. In still another aspect of some embodiments, the at least one cell feature is a boundary of a structure within the cell, a nuclear invagination, a nuclear shape, or a texture. In yet another aspect of some embodiments, the pre-selected total cell count number is at least 1500. In some embodiments, in which the sample is provided from a patient's lungs, the pre-selected cell count number reflects the number of bronchial epithelial cells (BECs) imaged.

In one or more embodiments, the method for digital cell pathology, further includes providing images of the cell to a user, wherein providing images comprises creating a Patient Background Information Gallery including data regarding the patient from whom the patient sample was obtained and displaying the Patient Background Information Gallery to the user. In another aspect of some embodiments, the data regarding the patient from whom the patient sample was obtained includes one or more of patient age, patient gender, patient prior history with cancer, and patient prior history with non-cancer diseases. In still another aspect of some embodiments, the method for digital cell pathology further includes analyzing at least one of the 2D or 3D images by AI-based cell characterization to characterize the cell as a type of normal cell, wherein the type of normal cell is squamous, macrophage, or columnar. In yet another aspect of some embodiments, the method for digital cell pathology further includes, when a cell is characterized as a type of normal cell increasing a total cell count for that type of normal cell by one, determining if the total cell count for that type of normal cell has reached a pre-selected total call count for that type of normal cell number, and, if not, returning to step a), or, if so, proceeding to step h).

In some embodiments of the method for digital cell pathology, the patient sample is obtained by isolating and preserving a plurality of cells from a sputum specimen. In another aspect of some embodiments, the patient sample comprises abnormal cells, bronchial epithelial cells (BECs), squamous cells, monocytes, lymphocytes, polymorphonuclear leukocytes, other white blood cells, debris, cell fragments, cell clusters and any combinations thereof. In still another aspect of some embodiments, the abnormal cells are selected from a group consisting of cells exhibiting atypia, dysplastic cells, pre-cancerous cells, pleomorphic parakeratosis, type II pneumocytes, abnormal squamous cells, adenocarcinoma cells, bronchioloalveolar carcinoma cells, abnormal neuroendocrine cells, small cell carcinoma cells, non-small cell carcinoma cells, tumor cells, neoplastic cells, bronchioloalveolar carcinoma cells, and any combination thereof. In yet another aspect of some embodiments, the method for digital cell pathology further includes, prior to (a), pre-processing the patient sample to stain the plurality of cells with an agent that facilitates generating the 2D images, the 3D image, or generating an AI-based characterization or user classification, or enriching the patient sample for cells of interest. Furthermore, in some embodiments, the method for digital cell pathology further includes, prior to (a), embedding the patient sample in an optical medium and injecting the optical medium with embedded sample into a capillary tube; and loading the capillary tube into the optical tomography system so that the capillary tube is between an illumination source and objective lens of the optical tomography system.

In one or more other embodiments, a digital cell classification system includes an optical tomography system and a processor. The optical tomography system is operable to generate a plurality of 2D images of a cell from a patient sample and generate a plurality of pseudo-projection images of the cell. The processor executes computer-executable instructions stored in a memory that cause the processor to: generate a 3D image of the cell using the pseudo-projection images; apply digital enhancement to at least one of the 2D or 3D images to improve determination of boundaries of structures with the cell, wherein the boundaries of structures with the cell provide indicia of abnormal features; analyze at least one of the 2D or 3D images using AI-based cell characterization and (i) features pre-determined by a human, wherein the analysis includes analyzing the boundaries of structures with the cell (ii) features determined by AI or other image components, or (iii) both to characterize the cell as normal or as having abnormal features, create a Normal Cell Gallery comprising images of cells characterized by AI-based characterization as normal; create a Diagnostic Cell Gallery with images of cells characterized by AI-based characterization as having abnormal features; display at least the Diagnostic Cell Gallery to a user; and record a user classification for at least one cell characterized by AI-based characterization as having abnormal features.

In some embodiments of the system for the digital cell classification system, the digital enhancement includes one or more of image sharpening, segmentation, contrast, scale bar overlay, brightness, gamma, color transformation, and opacity transformation. In another aspect of some embodiments, the analysis of the at least one of the 2D or 3D images by AI-based cell characterization includes aiding cell interpretation using patient age, patient gender, patient prior history with cancer, and patient prior history with non-cancer diseases. In still another aspect of some embodiments, the digital cell classification system further includes using the recorded user classification and data regarding the patient from whom the patient sample was obtained to generate an abnormality index for the patient sample. In yet another aspect of some embodiments, the digital cell classification system further includes comparing the abnormality index to a cancer threshold value and identifying the patient sample as positive or negative for cancer, based upon the comparison.

In one or more embodiments of the system for digital cell classification system, the at least one cell feature in the 3D image of a cell characterized by AI-based characterization as having abnormal features is identified by and highlighted in the 3D image by a processor. In another aspect of some embodiments, the at least one cell feature is highlighted by use of different colors for different cell features, decreasing the opacity of surrounding cell features, or both. In still another aspect of some embodiments, the at least one cell feature is a boundary of a structure within the cell, a nuclear invagination, a nuclear shape, or a texture. In yet another aspect of some embodiments, the digital cell classification system further includes providing images of the cell to a user, wherein providing images comprises creating a Patient Background Information Gallery including data regarding the patient from whom the patient sample was obtained and displaying the Patient Background Information Gallery to the user. Furthermore, in some embodiments, the data regarding the patient from whom the patient sample was obtained includes one or more of patient age, patient gender, patient prior history with cancer, and patient prior history with non-cancer diseases.

In some embodiments, the system for digital cell classification system further includes analyzing at least one of the 2D or 3D images by AI-based cell characterization to characterize the cell as a type of normal cell, wherein the type of normal cell is squamous, macrophage, or columnar. In another aspect of some embodiments, the patient sample comprises abnormal cells, BECs, squamous cells, monocytes, lymphocytes, polymorphonuclear leukocytes, other white blood cells, debris, cell fragments, cell clusters and any combinations thereof. In still another aspect of some embodiments, the abnormal cells are selected from a group consisting of cells exhibiting atypia, dysplastic cells, pre-cancerous cells, pleomorphic parakeratosis, type II pneumocytes, abnormal squamous cells, adenocarcinoma cells, bronchioloalveolar carcinoma cells, abnormal neuroendocrine cells, small cell carcinoma cells, non-small cell carcinoma cells, tumor cells, neoplastic cells, bronchioloalveolar carcinoma cells, and any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting and non-exhaustive embodiments are described with reference to the following drawings. In the drawings, like reference numerals refer to like parts throughout the various figures unless otherwise specified. For a better understanding of the present invention, reference will be made to the following Detailed Description, which is to be read in association with the accompanying drawings:

FIG. 1 is a schematic representation of an optical tomography system that may be used in the present disclosure;

FIG. 2 is a schematic representation of the optical tomography system as operated to acquire a plurality of 2D images of a cell that may be used in the present disclosure;

FIG. 3 is a logic diagram for a digital cell pathology method of the present disclosure;

FIG. 4 is a logic diagram for user display and cell classification method of the present disclosure;

FIG. 5 is an example normal cell gallery and 2D evaluation window according to the present disclosure;

FIG. 6 is an example diagnostic cell gallery and 2D evaluation window according to the present disclosure;

FIG. 7 is an example 2D image stack window according to the present disclosure;

FIG. 8 is an example set of 2D representations of 3D images of various cell types representative of those that may be used in methods of the present disclosure;

FIG. 9 is an example set of 2D representations of 3D images of an adenocarcinoma cell representative of those that may be used in methods of the present disclosure; and

FIG. 10 is an example 2D representation of a 3D visualization movie that may be used in methods of the present disclosure.

### DETAILED DESCRIPTION

The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks, have not been shown or described to avoid unnecessarily obscuring descriptions of the embodiments. Additionally, the various embodiments may be methods, systems, media, or devices. Accordingly, the various embodiments may be entirely hardware embodiments, entirely software embodiments, or embodiments combining software and hardware aspects.

Throughout the specification, claims, and drawings, the following terms take the meaning explicitly associated herein, unless the context clearly dictates otherwise. The term “herein” refers to the specification, claims, and drawings associated with the current application. The phrases “in one embodiment,” “in another embodiment,” “in various embodiments,” “in some embodiments,” “in other embodiments,” and other variations thereof refer to one or more features, structures, functions, limitations, or characteristics of the present disclosure, and are not limited to the same or different embodiments unless the context clearly dictates otherwise. As used herein, the term “or” is an inclusive “or” operator, and is equivalent to the phrases “A or B, or both” or “A or B or C, or any combination thereof,” and lists with additional elements are similarly treated. The term “based on” is not exclusive and allows for being based on additional features, functions, aspects, or limitations not described unless the context clearly dictates otherwise. In addition, throughout the specification, the meaning of “a,” “an,” and “the” include singular and plural references.

The present disclosure relates to a system and method for digital cell pathology. The system and method display and allow the classification of cells, particularly normal cells and potentially abnormal calls. The system and method may be used to detect cells indicative of cancer, such as lung cancer, among the cells having abnormal features. In some embodiments, lung cancer may be non-small cell lung cancer (NSCLC), particularly squamous carcinoma and adenocarcinoma. In some embodiments, lung cancer may be small cell lung cancer (SCLC). In other embodiments, particularly for the detection of other cancers or abnormal cells, samples may include urine (bladder cancers) or blood (circulating tumor cells (CTCs)).

The term “cancer” refers to a hyperproliferation of cells that results in unregulated growth, lack of differentiation, local tissue invasion, or metastasis.

The system and method may use various tools not available in traditional pathology methods, such as AI-based cell characterizations, enhanced images, image analysis tools, 3D images, and image storage and comparisons.

The system and method use optical tomography, such as current optical tomography systems, to detect cells and to generate 2D and 3D images of the cells.

In some embodiments, cells having abnormal features may include cells exhibiting atypia, dysplastic cells, pre-cancerous cells, pleomorphic parakeratosis, type II pneumocyte abnormal squamous cells, adenocarcinoma cells, bronchioloalveolar carcinoma cells, abnormal neuroendocrine cells, small cell carcinoma cells, non-small cell carcinoma cells, tumor cells, neoplastic cells, bronchioloalveolar carcinoma cells, and any combination thereof.

### Optical Tomography System

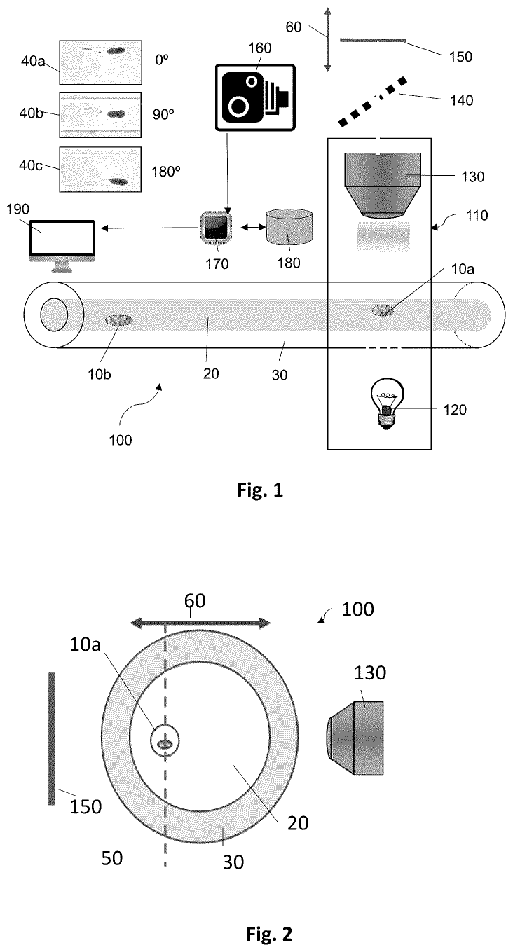

Referring now to FIG. 1 and FIG. 2, a system of the present disclosure may include, or a method of the present disclosure may be carried out using optical tomography system 100, which may be used to produce both 3D images and 2D images of a cell 10 (such as cell 10a or cell 10b). Although the operation of the optical tomography system is described for acquiring images of one cell 10, in a volume of optical medium in the optical path of a high-magnification microscope, images of multiple cells 10 within the same volume of optical medium may be acquired.

The optical tomography system 100 may include a cell imaging system 110, which includes an illumination source 120 optically coupled to an objective lens 130, such that illumination passes through the micro-capillary tube 30 and any intervening cell 10 before reaching the objective lens 130.

In embodiments disclosed herein, the optical tomography system 100 may be operated to generate 2D images of the cell 10. In such embodiments, the objective lens 130 has a focal plane 50 that moves as the objective lens 130 sweeps across the micro-capillary tube 30 and any cell 10 in a back-and-forth direction 60 to produce a plurality of 2D images (not shown). This method of generating 2D images by moving the objective lens 130 is different than the method of producing 3D images (described below) using pseudo-projection images that are generated by vibrating the mirror 150. The processor 170 is operable to receive the plurality of 2D images. In some embodiments, a 2D image of the central portion, such as the center of the cell 10 is designated to be a representative 2D image.

In some embodiments disclosed here, the optical tomography system 100 may be operated to generate 3D images of the cell 10, the illumination passes through the objective lens 130 to a beam-splitter 140, which causes part of the illumination to be deflected to a mirror 150 and reflected back to the beam-splitter 140 before being transmitted to a high-speed camera 160, and another part of the illumination to be transmitted directly through the beam-splitter 140 to the high-speed camera 160, to generate pseudo-projection images 40 of the cell 10 contained in an optical medium 20 in a micro-capillary tube 30.

During 3D imaging, at least one pseudo-projection image 40 of the cell 10 is generated by scanning the volume occupied by the cell 10 by vibrating mirror 150 in direction 60 (typically using an actuator, such as a piezo-electric motor, not shown), thus sweeping the plane of focus 50 through the cell 10 and then integrating the image to create the pseudo-projection image from a single perspective. Additional pseudo-projection images are obtained by rotating the micro-capillary tube 30. The pseudo-projection images are each a single image that represents a sampled volume that has an extent greater than the depth of field of the objective lens 130. The high-speed camera 160 generates, for each cell 10, a plurality of pseudo-projection images 40 that correspond to a plurality of axial micro-capillary tube rotation positions, examples of which are illustrated as 40a, 40b, and 40c in FIG. 1. In some embodiments, 500 pseudoprojection images are generated as the micro-capillary tube 30 is rotated through 360°.

In some embodiments, optical tomography system 100 is communicatively coupled to a processor 170 operable to receive the plurality of pseudo-projection images 40 from the high-speed camera 160 and use the pseudo-projection images 40 to generate a 3D image (not shown) of the cell 10.

Images before or after manipulation by the processor 170 may be stored in communicatively coupled memory 180. Patient Background Information, such as patient identifier data associated with the images and sample may be stored in communicatively coupled memory 180, as may patient health data.

In some embodiments, the processor 170 is then further operable to perform AI-based cell characterizations of the representative 2D image using 2D cell classifiers as described herein to determine if the cell 10 has abnormal features, and, after generating 3D images for a pre-selected number of normal cells, thereafter only generate 3D images of cells having abnormal features.

The processor 170 may send data regarding the cell 10, or data relating to or derived from a plurality of cells 10 contained in a patient sample to a communicatively coupled output 190. Data sent to the output 190 may include 2D or 3D images of one or more cells 10, or a summary of cells in a patient sample analyzed by the optical tomography system 100.

In some embodiments, the cell imaging system 110 includes the illumination source 120, the objective lens 130, the beam-splitter 140, the mirror 150, and the high-speed camera 160.

In some embodiments, the optical tomography system 100 further includes the processor 170, any communicatively coupled memory 180, and the communicatively coupled output 190.

In certain embodiments, the optical tomography system 100 further includes the micro-capillary tube 30, the optical medium 20, or one or more cells 10, but in other embodiments, the optical tomography system 100 does not include one or more of these potential components, although they may be supplied for operation of the system.

In certain embodiments, the optical tomography system 100 is operable to generate images in at least two distinct modes. One mode includes a cell search mode, during which a plurality of single-focal plane 2D images of a cell 10 are generated. 2D images in this mode are generated by moving the objective lens 130 in direction 60 to sweep the focal plane 50 through the cell. A second mode includes a projection image capture mode, during which pseudo-projection images 40 are generated and used to produce a 3D image of the cell 10. 3D images in this mode are generated by vibrating the mirror 150 in direction 60 to create the pseudo-projection images 40.

Cytology has been traditionally practiced using brightfield microscopy, where stained cells are smeared on a slide, and a coverslip is added. The pathologist locates a cell of interest, selects the desired magnification, adjusts focal depth and brightness, and examines the cell with the general cellular context based on the cannon of cytologic criteria. However, digital cell pathology provides several significant advantages for cell examination. For example, digital images may be enhanced beyond what is possible through standard microscopy through digital image processing.

### Digital Cell Pathology Methods

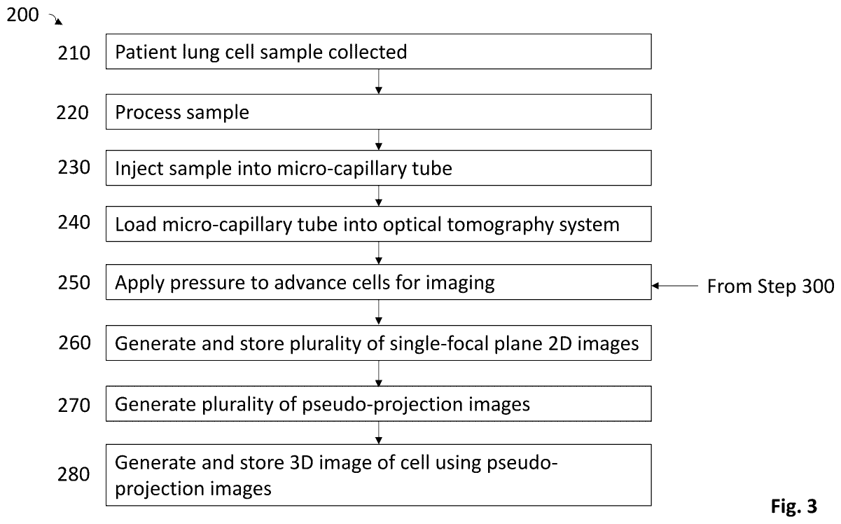

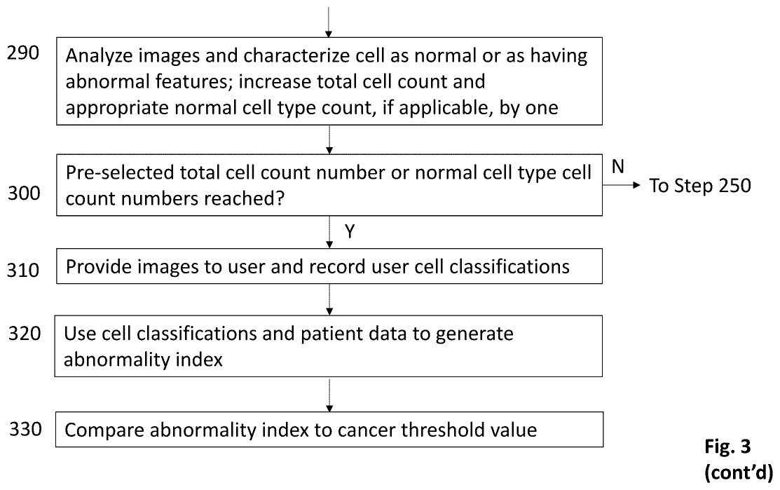

FIG. 3 describes a digital cell pathology method 200, which may be performed using the optical tomography system 100. Elements of the optical tomography system 100, as well as the processor 170, the memory 180, the output 190, and the input (not shown) are referenced in this description of the digital cell pathology method 200 as examples.

The digital cell pathology method 200 includes a step 210 in which a cell sample is collected from a patient. For example, the lung cell sample may be sputum, although other sample types, such as samples obtained by bronchoalveolar lavage (BAL), nasal swab, or biopsy may also be used. In some embodiments, particularly for the detection of other cancers, abnormal cells, samples bodily regions or issues, samples may, for example, include urine or blood. In other embodiments, the patient sample is previously collected or is collected by a third party, and thus, is now technically part of this digital cell pathology method 200.

Samples may undergo a digital cell pathology method 200 at any time during which cells are not to have degraded to the point where limited cellular content remains. The duration of such time may depend on the storage conditions, e.g., whether the sample is refrigerated or how or if the sample is processed. In some embodiments, the sample may undergo the digital cell pathology method 200 within 30 minutes, one hour, two hours, 6 hours, 12 hours, one day, two days, one week, or two weeks of collection.

In some embodiments, the sample contains abnormal cells, BECs, columnar cells, squamous cells, monocytes/macrophages, lymphocytes, polymorphonuclear leukocytes, other white blood cells, debris, cell fragments, cell clusters and any combinations thereof.

In step 220 the sample is processed for analysis. Processing may optionally include staining to render any of the plurality of cells 10 or any features, such as the nucleus of any of the plurality of cells 10, easier to detect using the optical tomography system 100, or otherwise staining or treating the cells with an agent that facilitates generating the plurality of 2D images of the cell 10, evaluating any of the plurality of 2D images, generating a 3D image of the cell 10, or analyzing the 3D image. In specific embodiments, the cells may be stained with hematoxylin.

Processing may optionally include, alone or in combination with staining, enrichment of the sample for cells of interest. For example, in some embodiments, the sample is enriched for BECs.

In one embodiment, the sample may be enriched for BECs by staining cytoskeleton proteins and using fluorescently activated cell sorting (FACS)-based enrichment.

In one embodiment, BEC enrichment may include treating the sample with at least one antibody, and typically a plurality of antibodies, having fluorescent conjugates that may be used for FACS-based enrichment. In particular, the antibodies may bind to BECs or they may bind contaminating inflammatory cells, such a neutrophils and macrophages. Antibodies that bind contaminating inflammatory cells may include anti-CD45 antibodies. In some embodiments, the sample is treated with a combination of antibodies that bind to BECs and antibodies that bind contaminating inflammatory cells, with the antibodies having distinct fluorescent conjugates. Antibodies that bind to BECs may bind to cytokeratins 8, 18 and 19 the BEC cytoskeleton.

In one embodiment, the cells may be stained with 4′,6-diamidino-2-phenylindole (DAPI) alone or in combination with antibodies for FACS-based cell enrichment purposes as well.

A specimen may be enriched for BECs by FACS in which gating is used to include BECs in the resulting specimen. Another approach involves depleting the specimen of undesirable cells stained with DAPI (which tend to be doublet cells or debris), high side-scatter objects, objects bound by anti-inflammatory cell antibodies, or any combinations thereof. In some embodiments, both gates to support enrichment for desired cells, such as BECs and depletion of undesirable cells, may be implemented concurrently or in series.

Following any optional staining or enrichment, the sample processing 220 includes placing the cells 10 contained in the sample in an optical medium 20.

The optical medium 20 may be any medium reasonably expected to maintain the cells 10 intact during the expected duration of time prior to and during the digital cell pathology method 200. The optical medium 20 may also have a viscosity that allows movement of the optical medium 20 through a micro-capillary tube 30. The optical medium 20 may also not interfere with image generation by the optical tomography system 100. In particular, the optical medium may have an optical index that matches the optical index of other components of the optical tomography system 100 and the micro-capillary tube 30 through which light passes during image acquisition. In some embodiments, the optical tomography system 100 components through which light passes and the micro-capillary tube 30 also have a matching index. For optimal optical tomography operation, any changes in light movement should be due to encountering the object to be imaged, not changes in the optical index of other components or objects in the light path.

In the step 230, the optical medium 20 containing a plurality of cells 10 from the patient sample is injected into a micro-capillary tube 30. In some embodiments, the micro-capillary tube 30 may have an outer diameter of 500 μm or less, for example, between 30 μm and 500 μm. In some embodiments, the micro-capillary tube 30 may have an inner diameter of 400 μm or less, for example, between 30 μm and 400μ M, such as 50 μm. In one embodiment, the entire portion of the patient sample to be analyzed is placed in one micro-capillary tube 30. In another embodiment, the portion of the patient sample to be analyzed is placed in a plurality of micro-capillary tubes 30, which may be evaluated sequentially. In still another embodiment, the sample may be pumped through the micro-capillary tube 30 from a sample reservoir.

In the step 240, the micro-capillary tube 30 is loaded into the optical tomography system 100, so that the micro-capillary tube 30 is between the illumination source 120, and the objective lens 130.

In step 250, the optical medium 20 and any cells 10 contained within it are advanced into (prior to the initial step 240) or through the micro-capillary tube 30 by applying pressure at one end of the micro-capillary tube, such that a different volume of the optical medium 20 carrying a different portion of the patient sample is in the optical path of the optical tomography system 100, between the illumination source 120 and the objective lens 130. In some embodiments, a plunger (not shown) is used to advance the optical medium 20. For instance a plunger may be applied to a reservoir (not shown) of the optical medium 20 and patient sample that is connected to the micro-capillary tube 30, forcing additional optical medium 20 from the reservoir into the micro-capillary tube 30.

Next, in step 260, the method generates and stores a plurality of single-focal plane 2D images of the cell 10 in cell search mode. In the cell search mode as used in step 260, the optical tomography system 100 generates 2D images of a cell 10. In some embodiments, in the cell search mode 310, the optical tomography system 100 sweeps the focal plane 50 through the cell 10 in the direction 60 at 1 μm intervals to capture a series of 2D images. To determine the location of and preserve the image of at least one cell 10, at least a portion of the series of single-focal plane 2D images are compiled and filtered by the processor 170 to determine if the 2D images contains features associated with a cell 10 or other solid object in the optical medium 20, such as being dark as compared to the optical medium 20. Multiple cells 10 may be identified in the same volume of optical medium 20 in the optical path of the optical tomography system 100.

In step 270, the method generates a plurality of pseudo-projection images of the cell 10 in projection image capture mode and, in step 280, generates and stores a 3D image of the cell 10 using the pseudo-projection images. In projection image capture mode, the optical tomography system 100 generates a plurality of pseudo-projection images 40 by vibrating the mirror 150. These images may be taken over 360 degrees of rotation around the cell 10. However, pseudo-projection 40 may be taken over as little as 180 degrees of rotation around the cell 10.

In step 280, the processor 170 uses at least a portion of the plurality of pseudo-projection images 40 to generate a 3D image of the cell 10. Typically all pseudo-projection images 40 are used to generate the 3D image of the cell 10. However, if pseudo-projection images 40 that are determined to be of poor quality or likely to contain errors, the 3D imaging of the cell may be discontinued. In addition, typically pseudo-projection images 40 that cover 360 degrees of rotation around the cell 10 are used to generate the 3D image, but pseudo-projection images that cover as little as 180 degrees of rotation around the cell 10 may be used.

In step 290, at least one of the 2D or 3D images is used to analyze the cell 10 and characterize the cell as normal or as having abnormal features. If the cell is characterized as normal, it may also be characterized as a certain normal cell type, typically squamous, macrophage, or columnar. After characterization, a total cell count is increased by one and a cell count for any applicable normal cell type is increased by one if the cell has been categorized as that normal cell type.

Characterization may be by AI-based cell characterization, in which the processor 170 may detect and measure any of a plurality of cell features in the 2D or 3D images. The cell features are pre-determined by a human. In some embodiments, the processor 170 detects a nucleus portion and a non-nucleus cellular portion of the cell 10 and segments 2D or 3D images into nucleus and non-nucleus portions. In some embodiments, the processor 170 detects and analyzes boundaries of other structures within the cell. Cell features may be in certain categories, including whole-cell, nucleus, cytoplasm, or nucleoli features. Cell features may be of certain types, such as greyscale histogram (e.g. e.t. median, average, 2^{nd}-3^{rd} or -4^{th} statistical moment), spatial distribution (e.g. statistical movements of the Fourier transform), shape (e.g. eccentricity, deviation from spherical ideal), volume, or ratio features (e.g. ratio of nucleus to cytoplasm volume, deviation of the nucleus from cell centroid and center of mass). In some embodiments, the cell feature measurements may include object shape features, cell-shape features, cytoplasm features, cell nucleoli features, distribution of chromatin, nuclear-shape features, nuclear-size features, such as area of nuclear surface, nuclear-texture features, nuclear invaginations, other morphometric elements, such as ratio of nuclear to cytoplasm volume, average grapy value, spatial frequencies, grey moments, geometric moments, or any combination thereof.

AI-based cell characterization may in addition or alternatively include analyzing features determined by AI (which may be the same or a different AI), or by analyzing other image components, which may also be determined by AI. Features analyzed in this embodiment may be the same as or different from cell features pre-determined by a human as discussed above.

Characterization of cells as a type of normal cell or as having abnormal features may have a pre-selected AI-based cell characterization accuracy for each normal cell type or for cells having abnormal features, such as a pre-selected AI-based cell characterization sensitivity or a pre-selected AI-based cell characterization specificity. These accuracy measures may differ from the accuracy of the completed assay, after evaluation of cells by a user.

For any process or assay, the “sensitivity” of the process or assay is defined as the percent of processed or assayed items (such as cells or the sample as a whole) that are actually positive for a property that are also correctly identified as positive by the process or assay.

For any process or assay the “specificity” of the process or assay is defined as the percent of processed or assayed items (such as cells or the sample as a whole) that are actually negative for a property that are also correctly identified as negative by the process or assay.

In the context of the categorization of cells or the overall assay, for each normal cell type or cells having abnormal features the correct identification of a cell for determining sensitivity or specificity may be what would be determined using a known identification method, such as microscope-based cytology (e.g. review or slides by a pathologist).

In some embodiments, the pre-selected AI-based cell characterization sensitivity for each type of normal cell or for cells having abnormal features may be at least 85%, at least 90%, at least 95%, or in a range of 85% to 100%, 85% to 99%, 85% to 95%, 85% to 90%, 90% to 100%, 90% to 99%, 90% to 95%, 95% to 100%, or 95% to 99%. In general, the normal cell AI-based cell characterization sensitivity may be set so that, given the prevalence of the cell type in the sample, the product of sensitivity prevalence yields the pre-selected number (e.g. four) of each type of normal cell for 99%, 99.9%, or 100% of samples.

In some embodiments, the pre-selected AI-based cell characterization specificity for each type of normal cell or for cells having abnormal features may be at least at least 60%, at least 65%, at least 70%, or in a range of 60% to 100%, 60% to 90%, 60% to 80%, 60% to 70%, 65% to 100%, 65% to 90%, 65% to 80%, 65% to 70%, 70% to 100%, 70% to 90%, or 70% to 80%. In general, the normal cell AI-based cell characterization specificity may be high, such that, for example, a nonsquamous cell is rarely identified as a squamous cell.

The assay as a whole may have similar sensitivities and specificities.

The assay as a whole may also have pre-selected cancer accuracy parameters. The cancer sensitivity is the percent of patients who actually have cancer who are identified as having cancer by the cancer detection method (a positive rest result).

In step 300, the total cell count and any applicable normal cell type counts are compared to pre-selected numbers to determine if the pre-selected number have been reached. If all pre-selected numbers have been reached, the method proceeds to step 310. If not, then method returns to step 250 to acquire images of additional cells.

In some embodiments, the pre-selected number of cells for each of the normal cell types may be at least two, at least three, at least four, at least five, at least ten, or two, three, four, five, or ten. In general, the sensitivity of any lung cancer detection method that uses 3D imaging of the type used in method 300 depends on the number of cells (particularly BECs) that are imaged. The pre-selected total cell count may, therefore, be at least 250, at least 400, at least 500 at least 600, at least 700, at least 800, at least 900, at least 1000, at least 1100, at least 1200, at least 1300, at least 1400, at least 1500, at least 2000, at least 2500, or in a range of 250 to 2500, 250 to 2000, 400 to 1900, 600 to 1800, 800 to 1700, 1000 to 1600, 1200 to 1600, or 1300 to 1500.

In some embodiments, more than one cell is located in a given volume of the optical medium, such that an additional cell may be identified, imaged, and used to update cell counts prior to the method proceeding to step 310 or returning to step 250.

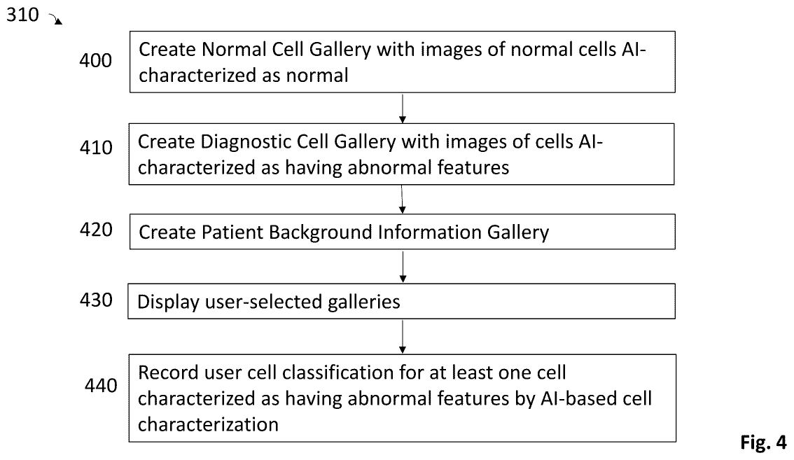

In step 310, images of at least one cell are provided to a user and at least one user cell classification is recorded. This process is described in further detail below in connection with FIG. 4.

In step 320, the processor uses cell classifications from step 310 with patient data to generate an abnormality index for the same that was analyzed.

Cell classifications may reflect a total number of cells classified as having abnormal features and a total cell count for cells analyzed, as well as the total numbers of cells AI-characterized as normal in total or by normal cell type. Cell classifications may also include predicted accuracy data. The abnormality index may be based, at least in part, on relative proportions of total cell numbers, particularly relative proportions of abnormal cells to other cells or total cells analyzed.

In some embodiments, patient data aids cell interpretation by the AI-based cell characterization system. In this manner, data may include one or more of the following: patient age, patient gender, patient prior history with cancer, and patient prior history with non-cancer diseases.

In step 330, the processor 170 compares that abnormality index to a cancer threshold value set to correspond with the patient sample being designated positive for cells indicative of cancer. This result may be provided to the user, for example using output 190, and may be stored, for example in memory 180.

Meyer, M. G., et al. (2015), The Cell-CT® 3-dimensional cell imaging technology platform enables the detection of lung cancer using the noninvasive LuCED sputum test. Cancer Cytopathology, 123:512-523 (doi.org/10.1002/cncy.21576); Wilbur, D. C., et al. (2015), Automated 3-dimensional morphologic analysis of sputum specimens for lung cancer detection: Performance characteristics support use in lung cancer screening. Cancer Cytopathology, 123:548-556 (doi.org/10.1002/cncy.21565); U.S. Pat. Nos. 6,519,355, 6,522,775, 6,591,003, 6,636,623, 6,697,508, 7,197,355, 7,494,809, 7,569,789, 7,738,945, 7,811,825, 7,835,561, 7,867,778, 7,787,112, 7,907,765, 7,933,010, 8,090,183, 8,155,420, 8,947,510, 9,594,072, 10,753,857, 11,069,054, and 20,200,018704, are each incorporated by reference herein in its entirety and specifically as it relates to the components, basic operation, including potential cell staining and enrichment, image formation, including formation of pseudo-projection images, and 3D classifiers of optical tomography systems and lung cancer detection methods and systems described herein.

### Image Galleries and User Analysis

FIG. 4 describes the steps of method step 310, in which images of at least one cell are provided to a user and at least one user cell classification is recorded.

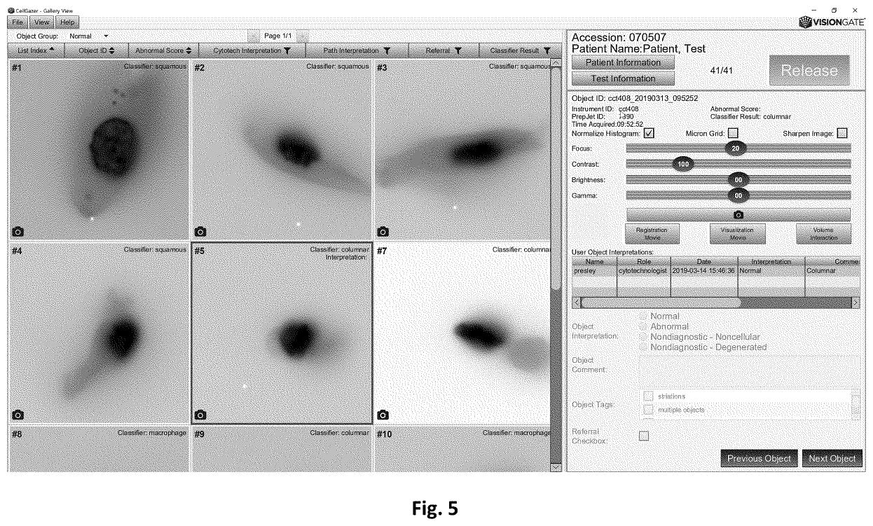

In step 400, the processor 170 creates an array of images of cells categorized by AI-based cell characterization as normal cells, which may be referred to as a “Normal Cell Gallery,” which may be displayed using output 190, for example using a video display. An example Normal Cell Gallery is provided in FIG. 5. The Normal Cell Gallery may include a representative 2D image of a cell and the AI-based cell characterization of the cell (e.g. the type of normal cell it was categorized as). The Normal Cell Gallery may contain representative images of a pre-selected number of each type of normal cell. In some embodiments, the user may be able to mark a cell in the Normal Cell Gallery as abnormal if it appears to be as such. In some embodiments, the user may be able to select a cell in the Normal Cell Gallery for replacement with images from another normal cell of the same type.

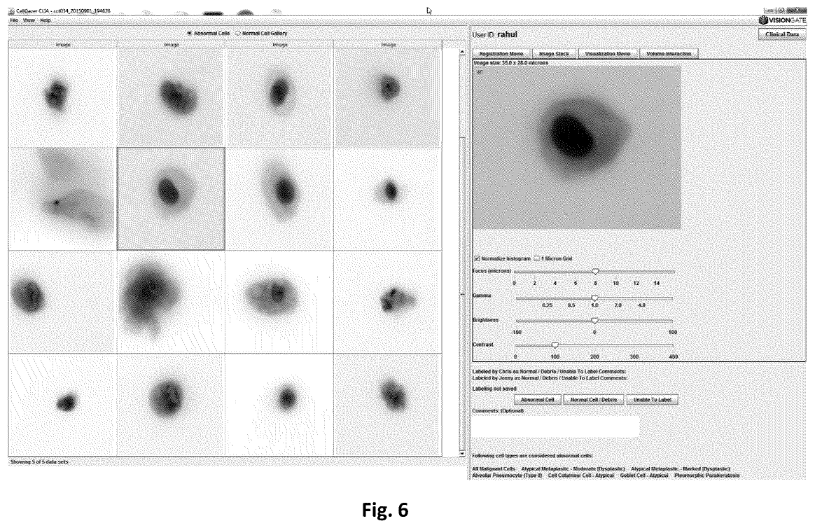

In step 410, which may occur, before, simultaneously with, or after step 400, the processor 170 creates an array of images of cells categorized by AI-based cell characterization as having abnormal features, which may be referred to as a Diagnostic Cell Gallery. An example Diagnostic Cell Gallery is provided in FIG. 6. In some embodiments, the Diagnostic Cell Gallery may include every cell categorized by AI-based cell characterization as having abnormal features. The Diagnostic Cell Gallery may include a representative 2D image of each cell.

In step 420, which may occur before, simultaneously with, or after step 400 or step 410, the processor 170 creates a Patient Background Information Gallery, which may include information that may be relevant to reviewing cells for abnormalities. For example, the Patient Background Information Gallery may include patient age, sex, prior history with cancer, non-cancer diseases, etc. This information may be helpful to normalize cell classification by the user. For example, cells may grow more prominent with age. Therefore, knowing patient age hedges against an erroneous classification upon finding larger than expected cell size.

In step 430, the Normal Cell Gallery, the Diagnostic Cell Gallery, or the Patient Background Cell Gallery is displayed if selected by the user. The user may select to display one or more of these galleries simultaneously and may move among galleries and among cells displayed in a gallery. This allows the user to compare cells having abnormal features to normal cells, review patient background data, and otherwise receive information relevant to cell classification. This comparison may be important because many diseases like the flu or COPD may activate cells. While cytologically normal, signs of activation may sway the diagnosis of a cell away from abnormality, whereas if these signs were not present in the normal cells, a cell may be more confidently identified as abnormal.

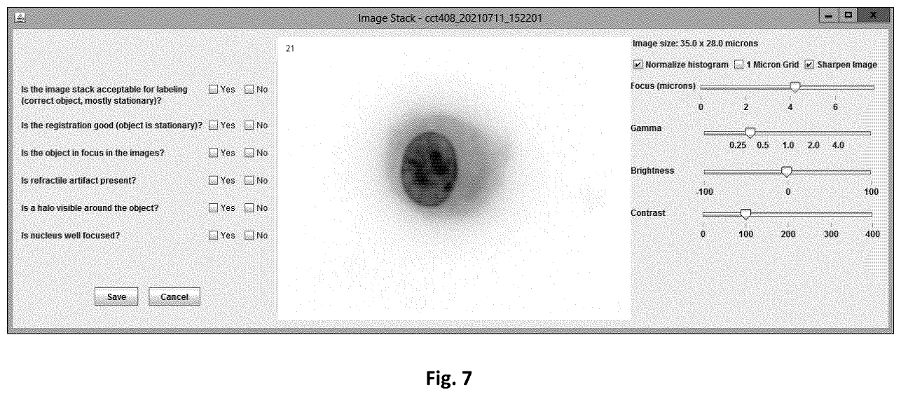

In some embodiments, a 2D image stack of a cell from the Normal Cell Gallery or Diagnostic Cell Gallery may be displayed in a window, such as illustrated in FIG. 7. Such a window may be displayed, for example, in response to the use pointing and clicking a cell in one of the galleries. The 2D image stack allows 2D analysis of the cell in a manner that is not possible with standard microscopy. For example, a standard microscope has a short focal range, and only portions of the cell in this range may be accurately viewed. Abnormalities in regions of the cell outside of a standard microscope focal range may be missed. The 2D image stack contains a plurality of in-focus 2D images that can and, in many embodiments, encompass the entire cell. The user may scroll through the 2D image stack image by image in any manner, but most often in a manner that replicates moving a microscope objective lens focus, as this approach is most familiar to many users. In addition, the user may adjust image properties, such as sharpness, gamma, brightness, and contrast, using controls such as those illustrated on the right side of FIG. 7.

Visual enhancements may also be applied to one or more of the images in the stack of 2D images. For example, a grid may be superimposed over the image to make it easier to spot or describe certain cell features. In addition, cell features selected by the user or identified as abnormal in the AI-based cell characterization may be identified and highlighted, for example using different colors or decreasing the opacity of surrounding features. Cell features may include nuclear invaginations, nuclear shape, and texture. Thus, identifying cells with abnormal features improves the final cell diagnosis.

Also as shown in FIG. 7, the user may be allowed or prompted to include annotations on image quality, which may be used in generating an abnormality index or in final classification of the sample as positive for cancer or negative for cancer.

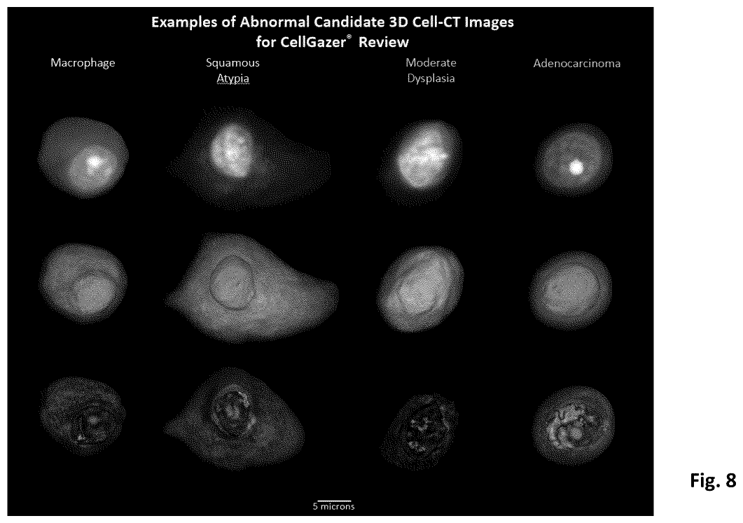

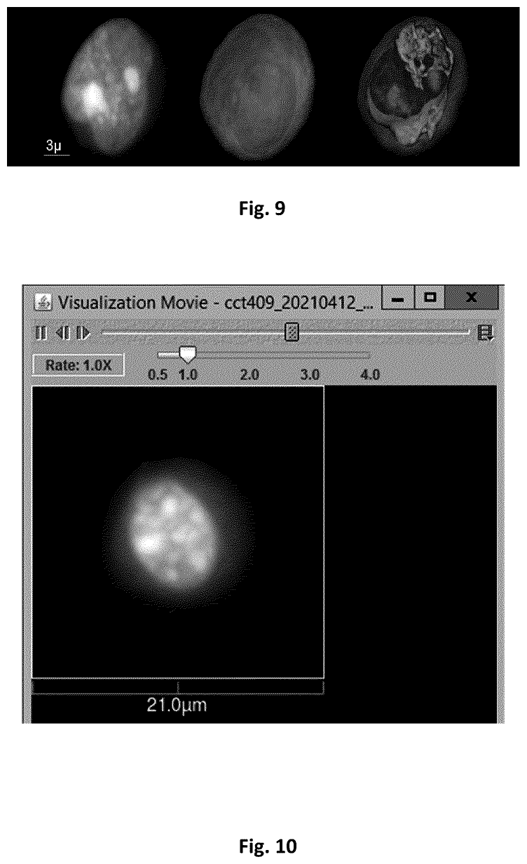

Even greater improvements in cytological analysis are provide by the 3D images, which may be displayed for each cell when selected by a user. 3D images may be pivoted to allow the cell to be viewed from any direction without loss of image resolution. This orientation-independent property is also known as isotrophic resolution. In contrast to isotrophic resolution, even a stack of 2D images provided limited information because a 2D image stack may be viewed in only one orientation from one direction. Example 2D representations of 3D images of the present disclosure are provided in FIG. 8 and FIG. 9. The 3D images may be in the form of a 3D visualization movie in which the user may virtually rotate the cell through 360° and establish 3D characteristics of the cell. A 2D image of such a 3D visualization movie is provided in FIG. 10. A 3D visualization movie, such as that in FIG. 10, may identify and focus on abnormal features identified in AI-based cell characterization.

The 3D visualization move may represent the cell as a series of voxels (3D pixels) in a bounding cube. The initial presentation of the 3D visualization move may be a maximum intensity projection, but the user may adjust to a more focused view, for example by using a volume interaction utility. The image may also be cropped to focus on a particular area, with a scale bar included if selected.

In some embodiments, the 3D image of a cell may exhibit super-resolution, that is, resolution at sizes smaller than the wavelength of light used to create the pseudo-projection images. For example, 3D image created using pseudoprojection images generated using 400 nm wavelength light may have a resolution of 200 nm. Super-resolution occurs because the substantial number of pseudo-projection images captured from a substantial number of different positions around the cell. For example, if 500 pseudo-projection images are captured, then each point in the cell is measured from 500 different perspectives, the overlap of which provides information over shorter distances than the wavelength of light used to generate the images.

Super-resolution facilitates the identification or measurement of cell features both during AI-based cell characterization and during evaluation by the user. The user may adjust image properties, such as sharpness, gamma, brightness, and contrast, using controls. Visual enhancements such as colors may also be applied. In addition, cell features selected by the user or identified as abnormal in the AI-based cell characterization may be identified and highlighted, for example using different colors or decreasing the opacity of surrounding features. For example, cell features, such as boundaries of the various structures within the cell, nuclear invaginations, nuclear shape, and texture can be identified and made apparent. Thus, 3D cytology using AI-based cell characterization can further inform cytology practice to draw relationships between morphology and disease.

In step 440, the user enters and processor 170 causes to be stored in memory 180 a cell classification at least one cell characterized as having abnormal features by AI-based cell characterization. In some embodiments, a cell classification is stored for each cell characterized as having abnormal features by AI-based cell characterization. A set data entry array, such as a set of check boxes corresponding to features of abnormal cells, may be used to enter the cell classification. An example set data entry array may be seen on the left side of FIG. 7.

FIG. 11 shows a system diagram that describes an example implementation of a computing system(s) for an optical tomography system 100 described herein. The functionality described herein for the optical tomography system 100, can be implemented either on dedicated hardware, as a software instance running on dedicated hardware, or as a virtualized function instantiated on an appropriate platform, e.g., a cloud infrastructure. In some embodiments, such functionality may be completely software-based and designed as cloud-native.

In particular, shown is an example computer system 1101. For example, such computer system 1101 may represent those in the control systems and other aspects described herein to implement the optical tomography system 100. In some embodiments, one or more special-purpose computing systems may be used to implement the functionality described herein. Accordingly, various embodiments described herein may be implemented in software, hardware, firmware, or in some combination thereof. Computer system 1101 may include memory 1102, one or more central processing units (CPUs) 1114, I/O interfaces 1118, other computer-readable media 1120, and network connections 1122.

Memory 1102 may include one or more various types of non-volatile and/or volatile storage technologies. Examples of memory 1102 may include, but are not limited to, flash memory, hard disk drives, optical drives, solid-state drives, various types of random-access memory (RAM), various types of read-only memory (ROM), other computer-readable storage media (also referred to as processor-readable storage media), or the like, or any combination thereof. Memory 1102 may be utilized to store information, including computer-readable instructions that are utilized by CPU 1114 to perform actions, including those of embodiments described herein.

Memory 1102 may have stored thereon control module(s) 1104. The control module(s) 1104 may be configured to implement and/or perform some or all of the functions of the systems, components and modules described herein for the optical tomography system 100. Memory 1102 may also store other programs and data 1110, which may include rules, databases, application programming interfaces (APIs), software platforms, cloud computing service software, network management software, network orchestrator software, network functions (NF), AI or ML programs or models to perform the functionality described herein, user interfaces, operating systems, other network management functions, other NFs, and the like.

Network connections 1122 are configured to communicate with other computing devices to facilitate the functionality described herein. In various embodiments, the network connections 1122 include transmitters and receivers (not illustrated), cellular telecommunication network equipment and interfaces, and/or other computer network equipment and interfaces to send and receive data as described herein, such as to send and receive instructions, commands and data to implement the processes described herein. I/O interfaces 1118 may include a video interface, other data input or output interfaces, or the like. Other computer-readable media 1120 may include other types of stationary or removable computer-readable media, such as removable flash drives, external hard drives, or the like.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A digital cell pathology method comprising:
a) generating, by an optical tomography system, a plurality of 2D images of a cell from a patient sample comprising a plurality of cells;
b) generating, by the optical tomography system, a plurality of pseudo-projection images of the cell;
c) generating a 3D image of the cell using the pseudo-projection images;
d) applying digital enhancement to at least one of the 2D or 3D images to improve determination of boundaries of structures with the cell, wherein the boundaries of structures, and their interrelationships of the structures, within the cell provide indicia of features to classify the cell;
e) analyzing cell features by AI-based cell characterization to characterize the cell as being a certain normal type or as having abnormal features by (i) analyzing features pre-determined by a human, wherein the analysis includes analyzing the boundaries of structures with the cell, (ii) analyzing features determined by AI or other image components, or (iii) both;
f) increasing a total cell count number by one;
g) determining if total cell count number has reached a pre-selected total cell count number and, if not, returning to step a);
h) if the total cell count number has reached the pre-selected number, providing images of the cell to a user, wherein providing images comprises:
creating a Normal Cell Gallery comprising images of cells characterized by AI-based characterization as normal;
creating a Diagnostic Cell Gallery with images of cells characterized by AI-based characterization as having abnormal features; and
displaying at least the Diagnostic and Normal Cell Galleries to the user; and
i) recording a user classification for at least one cell characterized by AI-based characterization as having abnormal features.

2. The method of claim 1, wherein the digital enhancement includes one or more of image sharpening, segmentation, contrast, scale bar overlay, brightness, gamma, color transformation, and opacity transformation.

3. The method of claim 1, wherein the analysis of the at least one of the 2D or 3D images by AI-based cell characterization includes aiding cell interpretation using patient age, patient gender, patient prior history with cancer, and patient prior history with non-cancer diseases.

4. The method of claim 1, further comprising using the recorded user classification and data regarding the patient from whom the patient sample was obtained to generate an abnormality index for the patient sample.

5. The method of claim 1, further comprising comparing the abnormality index to a cancer threshold value and identifying the patient sample as positive or negative for cancer based upon the comparison.

6. The method of claim 1, wherein at least one cell feature in the 3D image of a cell characterized by AI-based characterization as having abnormal features is identified by and highlighted in the 3D image by a processor.

7. The method of claim 6, wherein the at least one cell feature is highlighted by use of different colors for different cell features, decreasing the opacity of surrounding cell features, or both.

8. The method of claim 6, wherein the at least one cell feature is computed by finding a boundary of a structure within the cell.

9. The method of claim 1, further comprising analyzing at least one of the 2D or 3D images by AI-based cell characterization to characterize the cell as a type of normal cell, wherein the type of normal cell is squamous, macrophage, or columnar.

10. The method of claim 1, further comprising analyzing features computed from a cell image by AI-based cell characterization to characterize the cell as a type of normal cell, wherein the type of normal cell is normal bronchial epithelial, squamous, macrophage, or columnar.

11. A cell classification system comprising:
an optical tomography system operable to:
generate a plurality of 2D images of a cell from a patient sample; and
generate a plurality of pseudo-projection images of the cell; and
a processor that executes computer-executable instructions stored in a memory that cause the processor to:
generate a 3D image of the cell using the pseudo-projection images;
apply digital enhancement to at least one of the 2D or 3D images to improve determination of boundaries of structures with the cell, wherein the boundaries of structures with the cell provide indicia of abnormal features;
analyze at least one of the 2D or 3D images using AI-based cell characterization and (i) features pre-determined by a human, wherein the analysis includes analyzing the boundaries of structures with the cell (ii) features determined by AI or other image components, or (iii) both to characterize the cell as normal or as having abnormal features;
create a Normal Cell Gallery comprising images of cells characterized by AI-based characterization as normal; and
create a Diagnostic Cell Gallery with images of cells characterized by AI-based characterization as having abnormal features;
display at least the Diagnostic Cell Gallery to a user; and
record a user classification for at least one cell characterized by AI-based characterization as having abnormal features.

12. The method of claim 11, wherein the digital enhancement includes one or more of image sharpening, segmentation, contrast, scale bar overlay, brightness, gamma, color transformation, and opacity transformation.

13. The method of claim 11, wherein the analysis of the at least one of the 2D or 3D images by AI-based cell characterization includes aiding cell interpretation using patient age, patient gender, patient prior history with cancer, and patient prior history with non-cancer diseases.

14. The method of claim 11, further comprising using the recorded user classification and data regarding the patient from whom the patient sample was obtained to generate an abnormality index for the patient sample.

15. The method of claim 11, further comprising comparing the abnormality index to a cancer threshold value and identifying the patient sample as positive or negative for cancer based upon the comparison.

16. The method of claim 11, wherein at least one cell feature in the 3D image of a cell characterized by AI-based characterization as having abnormal features is identified by and highlighted in the 3D image by a processor.

17. The method of claim 16, wherein the at least one cell feature is highlighted by use of different colors for different cell features, decreasing the opacity of surrounding cell features, or both.

18. The method of claim 17, wherein the at least one cell feature is a boundary of a structure within the cell.

19. The method of claim 11, further comprising analyzing at least one of the 2D or 3D images by AI-based cell characterization to characterize the cell as a type of normal cell, wherein the type of normal cell is normal bronchial epithelial, squamous, macrophage, or columnar.

20. The method of claim 11, further comprising analyzing features computed from the 2D or 3D images by AI-based cell characterization to characterize the cell as a type of normal cell, wherein the type of normal cell is normal bronchial epithelial, squamous, macrophage, or columnar.
